(19) United States
(12) Patent Application Publication
(10) Pub. No.: US 2025/0136661 A1
(43) Pub. Date: May 1, 2025

(54) PHARMACEUTICAL COMPOSITION COMPRISING ADIPOSE TISSUE-DERIVED EXTRACELLULAR VESICLE AND BIOLOGIC AND USE OF PHARMACEUTICAL COMPOSITION FOR TREATING ARTHRITIS

(71) Applicant: GWO XI STEM CELL APPLIED TECHNOLOGY CO., LTD, Hsinchu County (TW)

(72) Inventors: Hsiu-Jung Liao, Hsinchu County (TW); Ssu-Jung Lu, Hsinchu County (TW); Yu-Chen Tsai, Hsinchu County (TW); Po-Cheng Lin, Hsinchu County (TW); Ming-Hsi Chuang, Hsinchu County (TW)

(21) Appl. No.: 18/525,926

(22) Filed: Dec. 1, 2023

(30) Foreign Application Priority Data
Oct. 31, 2023 (TW) 112141861

(51) Int. Cl. C07K 14/705; A61K 9/00; A61K 35/28; A61P 19/02; A61P 29/00

(52) U.S. Cl. CPC C07K 14/70575; A61K 9/0019; A61K 35/28; A61P 19/02; A61P 29/00; C07K 2319/30

(57) ABSTRACT

The present disclosure provides a pharmaceutical composition including an adipose tissue-derived extracellular vesicle and a biologic, and a use of the pharmaceutical composition for treating arthritis. The pharmaceutical composition of the present disclosure achieves the effect of treating arthritis through various efficacy experiments.

### CROSS-REFERENCE TO RELATED APPLICATION

This application claims priority of Taiwan patent application No. 112141861, filed on Oct. 31, 2023, the content of which is incorporated herein in its entirety by reference.

### BACKGROUND OF THE INVENTION

### 1. Field of the Invention

The present invention relates to a pharmaceutical composition comprising an adipose tissue-derived extracellular vesicle and a biologic, and use of the pharmaceutical composition for treating arthritis.

### 2. The Prior Art

Arthritis is one of the most common chronic diseases in the world. It is mainly caused by the deterioration of the cartilage of the joints or inflammation of the connective tissue, which causes joint pain and interferes with the normal movement of the joints. There are more than one hundred types in total. There are many causes of arthritis, and joint damage is also different. There are approximately 355 million arthritis patients in the world, of which more than 100 million are in China. In the United States, one in five people suffers from arthritis pain and discomfort; in Asia, one in six people suffers from the disease at some point in their lives. Arthritis is not a disease unique to the elderly, it can affect all ages, including children.

Arthritis can occur in the back, neck, knees, shoulder joints, hands, hip joints, and ankles. Most arthritis is related to human aging. People over sixty years old would suffer from some types of arthritis but young people would also suffer from arthritis. There are more than one hundred types of osteoarthritis (OA), rheumatoid, rheumatic and septic arthritis, traumatic osteoarthritis, and autoimmune arthritis, and ankylosing arthritis is also a type of arthritis. In particular, the autoimmune disease rheumatoid arthritis causes inflammation and destruction of joints. Excessive activation of osteoclasts would accelerate bone resorption.

At present, clinical drug treatments for arthritis have limited effect and have serious side effects (e.g., weight loss, infections, and allergic reactions), and many patients cannot continue to treat. More importantly, the drug only alleviates the symptoms, but fails to fundamentally solve the problem, so how to develop a new drug that can effectively treat arthritis is an important issue that the present invention intends to solve here.

In order to solve the above-mentioned problems, those skilled in the art urgently need to develop a novel pharmaceutical composition for treating arthritis for the benefit of a large group of people in need thereof.

### SUMMARY OF THE INVENTION

A primary objective of the present invention is to provide a pharmaceutical composition for treating arthritis, comprising an adipose tissue-derived extracellular vesicle and a biologic.

Another objective of the present invention is to provide a method for treating arthritis, comprising administering to a subject in need thereof a pharmaceutical composition comprising an effective amount of a combination of an adipose tissue-derived extracellular vesicle and a biologic.

According to an embodiment of the present invention, the adipose tissue-derived extracellular vesicle is an adipose tissue-derived mesenchymal stem cell (ADSC)-derived extracellular vesicle (EV).

According to an embodiment of the present invention, the arthritis is rheumatoid arthritis (RA).

According to an embodiment of the present invention, the pharmaceutical composition is in a dosage form for parenteral administration.

According to an embodiment of the present invention, the adipose tissue-derived mesenchymal stem cell (ADSC)-derived extracellular vesicle (EV) suppresses osteoclastogenesis.

According to an embodiment of the present invention, the adipose tissue-derived mesenchymal stem cell (ADSC)-derived extracellular vesicle (EV) suppresses signaling downstream of receptor activator of nuclear factor kappa-B (RANK).

According to an embodiment of the present invention, the adipose tissue-derived mesenchymal stem cell (ADSC)-derived extracellular vesicle (EV) inhibits receptor activator of nuclear factor kappa-B ligand (RANKL) stimulated calcium oscillations.

According to an embodiment of the present invention, the adipose tissue-derived mesenchymal stem cell (ADSC)-derived extracellular vesicle (EV) reduces RANKL expression of human T helper cells and aggressive synovial fibroblasts under inflammation.

According to an embodiment of the present invention, the combination of the adipose tissue-derived mesenchymal stem cell (ADSC)-derived extracellular vesicle (EV) and the biologic inhibits osteoclast differentiation and bone resorption in the subject.

According to an embodiment of the present invention, the biologic is Etanercept.

In summary, the pharmaceutical composition of the present invention achieves the effect on treating arthritis (particularly rheumatoid arthritis) by suppressing osteoclastogenesis, suppressing signaling downstream of RANK, inhibiting RANKL stimulated calcium oscillations, and reducing RANKL expression of human T helper cells and aggressive synovial fibroblasts under inflammation, and the combination of the adipose tissue-derived mesenchymal stem cell (ADSC)-derived extracellular vesicle (EV) and Etanercept inhibits osteoclast differentiation and bone resorption in the subject. It may become a new therapy for inflammation-induced osteoclast activation and inflammatory arthritis in the future.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIGS. 1A-1E show that the adipose tissue-derived mesenchymal stem cell (ADSC)-derived extracellular vesicle (EV) significantly inhibited osteoclast activation. (1A) Left and upper, transmission electron microscopy images showing the morphology of ADSC-EV. Left and lower, the size distribution of ADSC-EVs was determined by nanoparticle tracking analysis (NTA). Right, Western blot analysis of exosome markers (CD9, CD63, and CD81) and negative markers (Calnexin, Alix) in equivalent amounts of protein (10 μg) from ADSC-EV and ADSC cell lysates (as a control). (1B) Human peripheral blood mononuclear cell (PBMC) was plated in 96-well plates and stimulated with the receptor activator of nuclear factor kappa-B ligand (RANKL) (40 ng/ml)+macrophage colony-stimulating factor (M-CSF) (20 ng/ml) as indicated in the figure. After seven days, cells were analyzed for osteoclast differentiation. After incubation, cells were subjected to a Tartrate-resistant acid phosphatase (TRAP) assay. Cell morphology was examined by light microscopy (Scale bars, 100 μm), and the number of TRAP-positive multinuclear cells was quantified. (1C) Flow cytometric analyses of nuclear factor of activated T cell c1 (NFATc1)-expressing osteoclast precursor cells collected from RANKL-treated and RANKL+ADSC-EV-treated PBMCs. FSC represents forward scatter, and SSC represents side scatter. (1D) Whole-cell lysates were subjected to Western blot analysis with anti-NFATc1, c-Cathepsin K (c-CTSK), p-extracellular signal-regulated kinase (p-ERK), ERK, p-c-Jun N-terminal kinase (p-JNK), JNK, and receptor activator of nuclear factor kappa-B (RANK) antibodies. In addition, the glyceraldehyde-3-phosphate dehydrogenase (GAPDH) blot is shown as a loading control. (1E) Cathepsin K expression of RANKL-induced osteoclast differentiation suppressed by ADSC-EV. Human monocytes seeded on glass slips were stimulated with RANKL and M-CSF. The cells were fixed and exposed to anti-cathepsin K (green) and anti-F-actin (red) antibodies before being subjected to fluorescent-conjugated secondary antibodies. The cell nuclei were stained with DAPI (blue). The images were recorded using a confocal laser scanning microscope (Leica SP8 STED). The cells exposed to secondary antibodies only served as negative controls and demonstrated no fluorescent signals. One-way ANOVA was performed. *p<0.05, ** p<0.01, *** p<0.001, and **** p<0.0001 for treatments versus M-CSF or indicated groups.

FIGS. 2A-2E show the RNA sequencing analysis of ADSC-EV-treated human osteoclasts. (2A) Gene set enrichment analysis of DEG set (osteoclast activation, calcium signaling pathway, and negative regulation of response of cytokine stimulus) in ADSC-EV-treated human osteoclast precursors. (2B) Gene ontology analysis of RANKL-inducible genes regulated by ADSC-EV. (2C) Heatmap showing the gene expressions of the RANK-downstream signalings in ADSC-EV-treated human osteoclast precursors detected and analyzed by RNA sequencing. TNFRSF11A represents tumor necrosis factor receptor superfamily member 11A; CSF1R represents colony-stimulating factor-1 receptor; TREM2 represents triggering receptor expressed on myeloid cells 2; OSCAR represents osteoclast-associated receptor; MAPK1 represents mitogen activated protein kinase 1; CBL1 represents Calcineurin B-like protein 1; NFATC1 represents nuclear factor of activated T cell c1; CTSK represents Cathepsin K; SRC represents SRC proto-oncogene; SYK represents spleen tyrosine kinase; TRAF6 represents tumor necrosis factor-receptor-associated factor-6; BTK represents Bruton's tyrosine kinase; PLCG2 represents phospholipase C gamma 2; CALM1 represents calmodulin 1. (2D) The osteoclast precursors were subjected to western blot analysis with antibodies specific to RANK, C-CBL, PI3K, p-SRC, SRC, and GAPDH. GAPDH was used as a loading control. (2E) miR 143-3p was highly detected in ADSC-EV. qRT-PCR analysis showed that miR 143-3p mRNA expression levels significantly increased in ADSC-EV. Mean±SD, n=3 biologically independent samples, *** P<0.001 by Student's t-test. ADSC-EV modulated the RANKL-induced downstream signaling pathways to inhibit osteoclastic differentiation from human osteoclast precursors.

FIGS. 3A and 3B show that ADSC-EV dampened RANKL stimulated calcium oscillations during osteoclast differentiation. (3A) ADSC-EV suppressed Ca²⁺ imaging of RANKL/M-CSF-stimulated osteoclast precursors. The left shows Mag-Fura-4 images of recorded cells analyzed using confocal microscopy (Leica). The right quantifies the Mag-Fura-4 fluorescence ratio in single cells treated with RANKL and ADSC-EV for 120 h. (3B) Protein expression levels of cAMP-response element binding protein (CREB) and p-CREB proteins during osteoclast differentiation in human monocytes treated with RANKL/M-CSF and ADSC-EV for 5 days.

FIGS. 4A-4F show that ADSC-EV significantly suppressed RANKL expression on the human T helper cell 17 (Th17) cells and synovial fibroblasts. (4A) Flow cytometric data showing the percentage of expressing IL-17+CD4+ populations were significantly decreased by ADSC-EV treatment at day 5. (4B) Quantification of the IL-17⁺CD4⁺ populations by ADSC-EV. (4C) Expression and mean fluorescence intensity (MFI) of RANKL in IL-17⁺CD4⁺ T cells. For MFI and percentages, at least 1000 IL-17⁺CD4⁺ T cells were quantified. (4D) Quantification of the RANKL expression on the IL-17⁺CD4⁺ T populations by ADSC-EV. (4E) IL-17A and IL-22 production were suppressed by ADSC-EV using ELISA assay. (4F) ADSC-EV suppressed genes of inflammation. Analysis of mRNA expression in ADSC-EV treated with LPS-induced synovial fibroblast inflammation. We compared these gene expressions treated with LPS (2 μg/ml) and ADSC-EV (1×10¹¹ particle/ml) for 48 hrs.

The cells were harvested, and RNA was extracted to determine gene expression levels of cytokines by using a LightCycler® (Roche Applied Science). The fold change of gene expressions in the y-axis was calculated based on the same gene expression in untreated synovial fibroblasts. One-way ANOVA was performed. * p<0.05, ** p<0.01, *** p<0.001, and **** p<0.0001 for treatments versus Th17 Skewing or indicated groups.

FIGS. 5A-5F show that combination of ADSC-EV and etanercept inhibited bone resorption in type II collagen-induced model (CIA) rats. (5A) Photomicrographs from the hind paw of CIA rats depict severe arthritis and swollen lymph nodes. ADSC-EV inhibited the local swelling of the hindfoot and popliteal lymph nodes in the RA joints. (5B) The body weight and thickness of the pads for each group were by the number of arthritic rats to calculate a mean (±SEM). A combination of ADSC-EV and etanercept reversed the body weight. Hind paw thickness of CIA rats graphs showed a combination of ADSC-EV and etanercept reduced the swelling of the hind paws. (5C) μCT imaging analysis of the hind paws and femurs. Representative three-dimensional μCT reconstruction images showed that ADSC-EV inhibited bone erosion in hind paws and femurs. (5D) The bar charts indicated femoral trabecular bone mass quantification with bone volume, bone surface density, and trabecular number of sham or rats treated with ADSC-EV and suppressed the decrease of bone loss. (5E) TRAP staining of hind paws on paraffin-embedded bone sections in sham, ADSC-EV, and Etanercept-treated rats (scale bar, 300 μm; red-purple color to TRAP-positive osteoclasts) are shown on the right. (5F) Lymph nodes of indicated rats were harvested, and cells were analyzed by fluorescence activated cell sorter (FACS). Cells were gated on CD4⁺ cells, and the expression of RANKL was monitored. In addition, the percentage of CD4⁺RANKL⁺ in the live cell gate was determined. Data are given as mean (±SEM) of three independent experiments. n=6.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, the term “adipose tissue-derived mesenchymal stem cell (ADSC)” refers to mesemchymal stem cells separated from fat, which are multipotent stem cells having high plasticity. After induction, they can be differentiated into cells of many different tissues.

According to the present invention, the extracellular vesicle comprises exosome and microvesicles.

As used herein, the term “treating” or “treatment” refers to alleviating, reducing, ameliorating, relieving, or controlling one or more clinical signs of a disease or disorder, and lowering, stopping, or reversing the progression of severity regarding the condition or symptom being treated.

According to the present invention, the pharmaceutical composition can be manufactured to a dosage form suitable for parenteral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, and intralesional injection.

The pharmaceutical composition according to the present invention can comprise a pharmaceutically acceptable carrier which is widely used in pharmaceutical manufacturing technology. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar solution, aqueous solution containing alcohol, and combinations thereof.

The experimental animals used in the following examples are described as follows. The Sprague-Dawley (SD) rats (male, 6 weeks old) were purchased from BioLASCO. The experimental animals were bred and maintained under specific pathogen-free conditions in the Far Eastern Memorial Hospital animal care unit. All animal work was conducted according to the Association for Assessment and Accreditation of Laboratory Animal Care guidelines. In addition, all animal experiments were approved by the Animal Ethics Committee of the Far Eastern Memorial Hospital (IACUC number: IACUC-2022 (4)-MOST-09).

The procedure regarding induction and evaluation of collagen-induced arthritis (CIA) is as follows. Chicken CII (200 μg/rat; Chondrex, cat. no. 20022) was emulsified in equal volumes of complete Freund's adjuvant (Chondrex, cat. no. 7009) on ice to obtain 1 mg/ml solution in male SD rats. Each rat in the CIA group was injected intradermally at the base of the tail with 100 μl of a solution. In contrast, animals in the control group received an equal volume of physiological saline. Immunization was repeated 21 days after the first injection. Arthritis development was monitored and scored blinded every second or third day. The development of arthritis was monitored, and the arthritis score was evaluated every 2 days. The level of inflammation for each paw was graded from 0 to 4 by the following scale: 0=absence of inflammation, 1=paw with detectable swelling in a single digit, 2=paw with swelling in more than one digit, 3=paw with swelling of all digits and instep, and 4=severe swelling of the paw and ankle. The arthritic scores of four paws were recorded. Hind paws of CIA rats were subjected to μCT analysis.

The procedure regarding RNA sequencing and analysis is as follows. We used STAR (v2.7.3a) two-pass mapping strategy to align the raw FASTQ reads against the human reference genome (GRCh 38) downloaded from the Ensembl database. We used DESeq2 (v1.26) to normalize the raw read counts quantified by STAR with the human gencode v32 gene annotation. We used |log 2 fold change|>1 and adjusted p-value<0.05 to get differential expression genes, and then used the gene list to perform gene ontology (GO) and Kyoto Encyclopedia of Genes and Genomes (KEGG) analysis by STRING database (v11.5). To perform gene set enrichment analysis (GSEA), we first converted gene expression data into the pre-ranked format by calculating the log 2 fold-change of different conditions. Next, we used the GSEA software (v4.1.0) and MSigDB v7.2 with pre-rank mode to calculate the normalized enrichment scores and false discovery rate (FDR).

The procedure regarding osteoclast differentiation in vitro is as follows. We used human peripheral blood mononuclear cell (PBMC) as model systems of osteoclastogenesis. The cell type differentiates into osteoclast-like cells in the presence of receptor activator of nuclear factor kappa-B ligand (RANKL) (Biolegend, cat. no. 591102) plus macrophage colony-stimulating factor (M-CSF) (Biolegend, cat. no. 574802). The human PBMC cells were cultured in minimum essential medium (MEM) supplemented with 20 ng/ml M-CSF, 10% FCS, 2 nM L-glutamine, and 100 U/ml penicillin/streptomycin for three days. Adherent cells were referred to as monocytes. For induction of osteoclast differentiation, PBMC were seeded in a 96-well plate at a density of 2× 105/cells and cultured for seven days in the presence of 50 ng/ml RANKL and 20 ng/ml M-CSF.

The procedure regarding Tartrate-resistant acid phosphatase (TRAP) staining in cells and bone tissue is as follows. Cells were fixed and stained with TRAP (Sigma-Aldrich, cat. no. 387A) for 1 h at 37° C., then counter-staining with a hematoxylin solution. TRAP⁺ multinuclear cells (MNCs) (with more than three nuclei) were regarded as osteoclasts and counted under an inverted phase-contrast microscope. The morphological features of osteoclasts were photographed with a photomicroscope. To detect the osteoclasts in the tissue sections, osteoclast numbers were measured by quantifying cells that were positively stained for TRAP. Briefly, specimens were fixed for the 30 s and then stained with naphthol AS-BI phosphate and a tartrate solution for 1 h at 37° C., followed by counter-staining with a hematoxylin solution. TRAP-positive multinuclear cells with three or more nuclei were considered osteoclasts and counted under an inverted-phase contrast microscope. In addition, the total number of TRAP-positive cells and the number of nuclei per TRAP-positive cell in each well were counted.

Bones were fixed for 24 h in 4% formalin and decalcified in ethylenediaminetetraacetic acid (EDTA) (Calbiochem, CAS. no. 6381-92-6). Serial paraffin sections (4 μm) were stained for TRAP using a Leukocyte Acid Phosphatase Kit (Sigma-Aldrich, cat. no. 387A) according to the manufacturer's instructions or with hematoxylin-eosin. All analyses were performed using a microscope (Nikon) with a digital camera and an image analysis system for performing histomorphometry (Osteomeasure; OsteoMetrics).

The procedure regarding immunoblot analysis is as follows. Total cell extracts were obtained using lysis buffer containing 150 mM Tris-HCl (pH 6.8), 6% SDS, 30% glycerol, and 0.03% Bromophenol Blue; 10% 2-ME was added immediately before harvesting cells. Cell lysates were fractionated on 10% SDS-PAGE, transferred to Immobilon-P membranes (Millipore), and incubated with specific antibodies. Western Lightning plus-ECL (PerkinElmer) was used for detection. nuclear factor of activated T cell c1 (NFATc1) antibody (cat. no. 4389, 1:1000) was from Cell signaling; Cathepsin K ([3F9] (cat. no. ab37259), Abcam, 1:1000), SRC [32G6] (cat. no. 2123, Cell signaling, 1:1000), p-SRC (cat. no. 2101, Cell signaling, 1:1000), C-Casitas B-lineage lymphoma (c-cbl) (cat. no. 2747, Cell signaling, 1:1000), cAMP-response element binding protein (CREB) (cat. no. ab31387, Abcam, 1:2000) and CREB phospho S129⁺$133 (cat. no. ab10564, Abcam, 1:2000) antibodies.

The procedure regarding immunofluorescence staining is as follows. Preosteoclasts were fixed with 4% PFA (pH 7.2) in PBS for 30 min at R/T. Fixed samples were then permeabilized for 30 min with 0.5% Triton X-100 in PBS and blocked for 1 h with 3% BSA in 0.1% Triton X-100 in PBS. Next, samples were incubated for 24 hours with primary antibodies against Cathepsin K (1:50, Abcam) at 4° C., followed by 1 h incubation at R/T with the Alexa-fluor-488 conjugated secondary antibodies (goat-anti-mouse for Cathepsin K, 10 μg/ml). Then, the samples were incubated with Alexa-fluor-568-labelled F-actin antibody (1:200) for 30 min at R/T. Fluorescence images were acquired with a Leica SP8 STED confocal microscope.

The procedure regarding calcium oscillation measurements is as follows. Human osteoclast precursors were loaded with 5 μM Mag-Fluo-4 AM and analyzed using confocal microscopy (Leica). Cells were excited at 488 nm, and emissions at 500 to 550 nm for Mag-Fluo-4 AM and 600 to 680 nm for Fura Red were acquired simultaneously at 3-second intervals. The relative intracellular calcium levels in single cells were monitored for 300 seconds and indicated by the fluorescence intensity of Mag-Fluo-4 AM. H₂O₂ (1 mM) was used to confirm cellular responsiveness to calcium influx for every experiment. At least 100 single cells in each condition were monitored, and each condition was repeated at least 3 times.

The procedure regarding gene expression profiles of osteoclastogenesis-quantitative real-time PCR is as follows. The osteoclast-like cells were collected and extracted to determine gene expression levels of osteoclastogenic markers using a LightCycler® (Roche Applied Science). The fold change of gene expression in the y-axis was calculated based on the same gene expression in untreated cells.

The procedure regarding statistical analysis is as follows. The data were presented as mean±SEM and analyzed using GraphPad Prism software (Version 10.0). One-way ANOVA calculated P values for multiple comparisons using the indicated post hoc Bonferroni test with at least three replicates. In addition, two groups were compared; a non-paired Student's t-test was used; in vivo data was analyzed using two-way ANOVA.

### Example 1

### Adipose Tissue-Derived Mesenchymal Stem Cell (ADSC)-Derived Extracellular Vesicle (EV) Strongly Suppressed Osteoclastogenesis

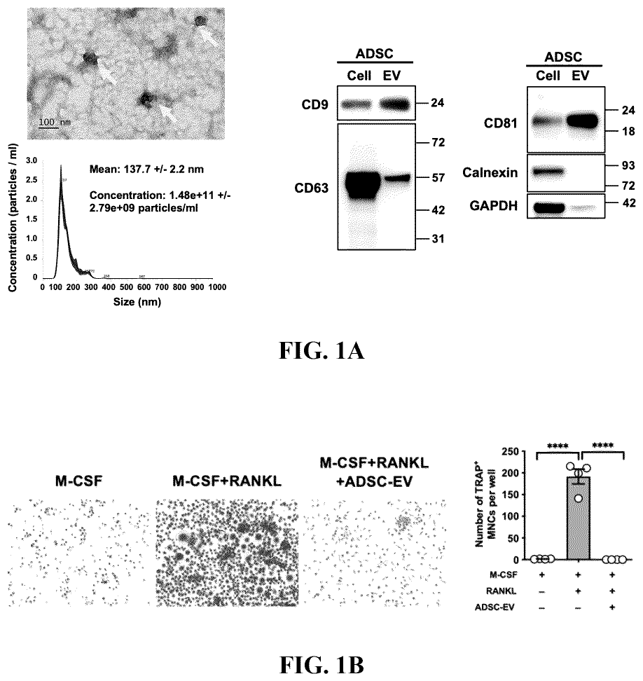

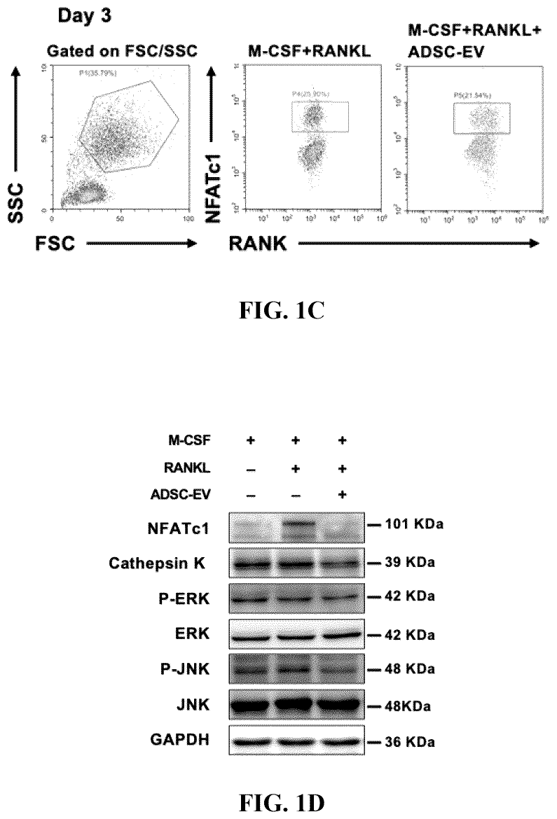

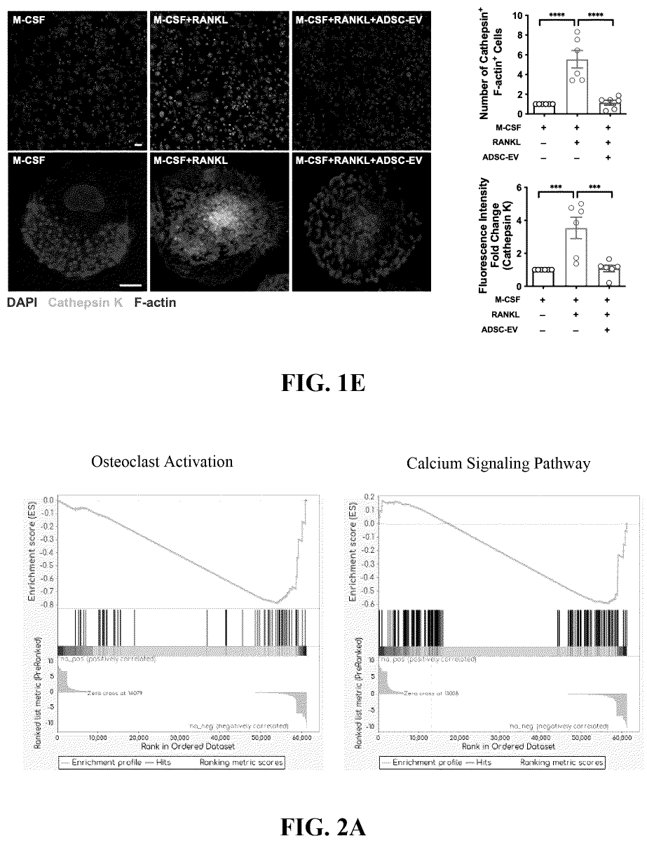

FIGS. 1A-1E show that the adipose tissue-derived mesenchymal stem cell (ADSC)-derived extracellular vesicle (EV) significantly inhibited osteoclast activation. (1A) Left and upper, transmission electron microscopy images showing the morphology of ADSC-EV. Left and lower, the size distribution of ADSC-EVs was determined by nanoparticle tracking analysis (NTA). Right, Western blot analysis of exosome markers (CD9, CD63, and CD81) and negative markers (Calnexin, Alix) in equivalent amounts of protein (10 μg) from ADSC-EV and ADSC cell lysates (as a control). (1B) Human peripheral blood mononuclear cell (PBMC) was plated in 96-well plates and stimulated with the receptor activator of nuclear factor kappa-B ligand (RANKL) (40 ng/ml)+macrophage colony-stimulating factor (M-CSF) (20 ng/ml) as indicated in the figure. After seven days, cells were analyzed for osteoclast differentiation. After incubation, cells were subjected to a Tartrate-resistant acid phosphatase (TRAP) assay. Cell morphology was examined by light microscopy (Scale bars, 100 μm), and the number of TRAP-positive multinuclear cells was quantified. (1C) Flow cytometric analyses of nuclear factor of activated T cell c1 (NFATc1)-expressing osteoclast precursor cells collected from RANKL-treated and RANKL+ADSC-EV-treated PBMCs. FSC represents forward scatter, and SSC represents side scatter. (1D) Whole-cell lysates were subjected to Western blot analysis with anti-NFATc1, c-Cathepsin K (c-CTSK), p-extracellular signal-regulated kinase (p-ERK), ERK, p-c-Jun N-terminal kinase (p-JNK),-JNK, and receptor activator of nuclear factor kappa-B (RANK) antibodies. In addition, the glyceraldehyde-3-phosphate dehydrogenase (GAPDH) blot is shown as a loading control. (1E) Cathepsin K expression of RANKL-induced osteoclast differentiation suppressed by ADSC-EV. Human monocytes seeded on glass slips were stimulated with RANKL and M-CSF. The cells were fixed and exposed to anti-cathepsin K (green) and anti-F-actin (red) antibodies before being subjected to fluorescent-conjugated secondary antibodies. The cell nuclei were stained with DAPI (blue). The images were recorded using a confocal laser scanning microscope (Leica SP8 STED). The cells exposed to secondary antibodies only served as negative controls and demonstrated no fluorescent signals. One-way ANOVA was performed. *p<0.05, ** p<0.01, *** p<0.001, and **** p<0.0001 for treatments versus M-CSF or indicated groups.

We first analyzed human ADSC-EV using nanoparticle tracking analysis (NTA) to analyze the sizes and concentrations and transmission electron microscopy (TEM) to examine the morphology (FIG. 1A, left, upper). We found EV sizes and concentrations (FIG. 1A, left, lower). Then, we did Western Blotting using the exosome markers CD9, CD63, and CD81. The results showed that CD9, CD63, and CD81 were expressed in ADSC-EV (FIG. 1A, right). To test our hypothesis that ADSC-EV acts with immunomodulatory function on osteoclast formation, we employed ADSC-EV on human peripheral blood mononuclear cells (PMBCs) to determine whether ADSC-EV directly impacts osteoclast differentiation. M-CSF and RANKL treatment resulted in notably greater osteoclast number and size in human PBMCs via staining for TRAP (tartrate-resistant acid phosphatase), a marker for mature osteoclasts. Conversely, ADSC-EV treatment resulted in a lower number of TRAP⁺ smaller-sized osteoclasts (FIG. 1B). To explore the underlying RANK-downstream signaling mechanism for the immunomodulation of ADSC-EV, human PMBCs were isolated and induced under osteoclast lineage cell polarization conditions in vitro. ADSC-EV was added during differentiation into osteoclast precursors; the results showed that ADSC-EV treatment inhibited the NFATc1 expression in the RANK-expressing osteoclast precursors significantly (FIG. 1C), accompanied by decreasing the protein levels of NFATc1, cathepsin K, p-ERK, ERK, p-JNK, JNK as assessed by Western blotting (FIG. 1D). Cathepsin K is a cysteine proteinase expressed predominantly and responsible for the degradation of type I collagen in mature osteoclast-mediated bone resorption. Confocal immunofluorescence imaging confirmed the inhibitory effect of cathepsin K, showing that F-actin and cathepsin K expressions in the human osteoclast precursors were suppressed about three-fold in ADSC-EV treatment compared with the stimulation of RANKL and M-CSF (FIG. 1E).

### Example 2

### ADSC-EV Suppressed Signaling Downstream of Receptor Activator of Nuclear Factor Kappa-B (RANK)

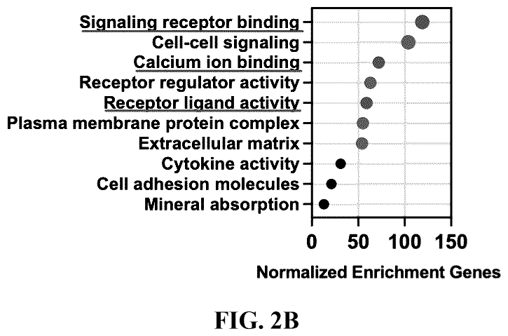

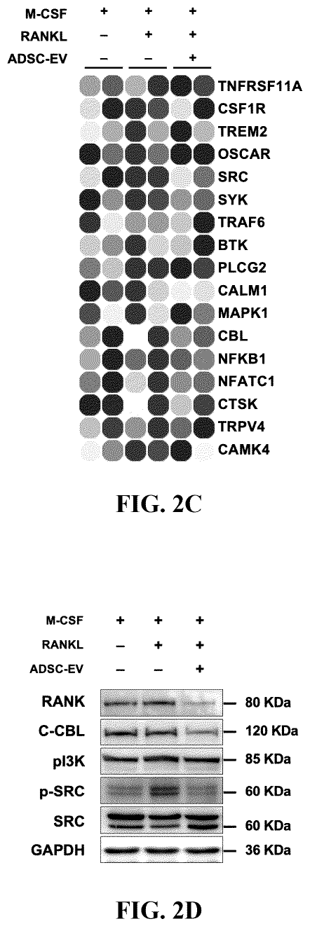

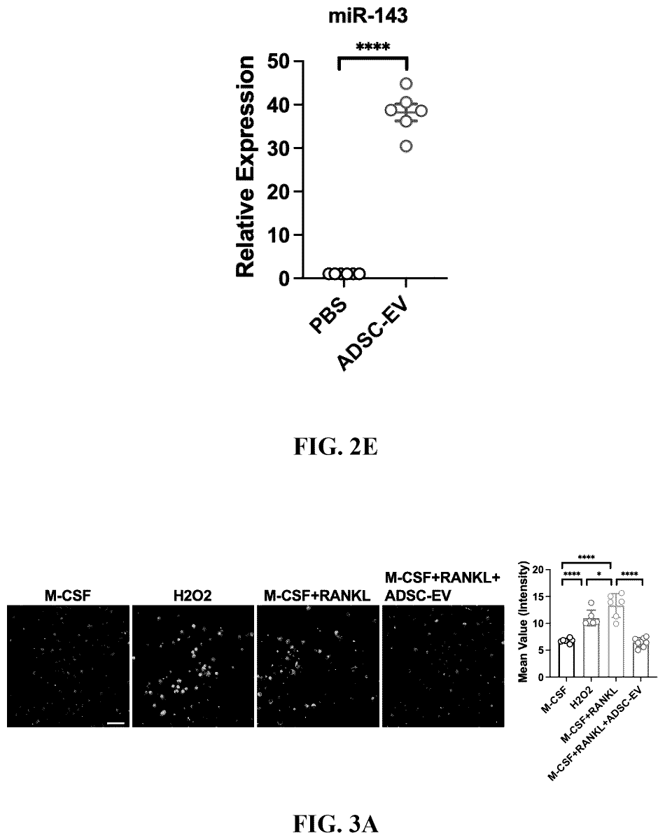

FIGS. 2A-2E show the RNA sequencing analysis of ADSC-EV-treated human osteoclasts. (2A) Gene set enrichment analysis of DEG set (osteoclast activation, calcium signaling pathway, and negative regulation of response of cytokine stimulus) in ADSC-EV-treated human osteoclast precursors. (2B) Gene ontology analysis of RANKL-inducible genes regulated by ADSC-EV. (2C) Heatmap showing the gene expressions of the RANK-downstream signalings in ADSC-EV-treated human osteoclast precursors detected and analyzed by RNA sequencing. TNFRSF11A represents tumor necrosis factor receptor superfamily member 11A; CSF1R represents colony-stimulating factor-1 receptor; TREM2 represents triggering receptor expressed on myeloid cells 2; OSCAR represents osteoclast-associated receptor; MAPK1 represents mitogen activated protein kinase 1; CBL1 represents Calcineurin B-like protein 1; NFATC1 represents nuclear factor of activated T cell c1; CTSK represents Cathepsin K; SRC represents sarcoma; SYK represents spleen tyrosine kinase; TRAF6 represents tumor necrosis factor-receptor-associated factor-6; BTK represents Bruton's tyrosine kinase; PLCG2 represents phospholipase C gamma 2; CALM1 represents calmodulin 1. (2D) The osteoclast precursors were subjected to western blot analysis with antibodies specific to RANK, C-CBL, PI3K, p-SRC, SRC, and GAPDH. GAPDH was used as a loading control. (2E) miR 143-3p was highly detected in ADSC-EV. qRT-PCR analysis showed that miR 143-3p mRNA expression levels significantly increased in ADSC-EV. Mean±SD, n=3 biologically independent samples, *** P<0.001 by Student's t-test. ADSC-EV modulated the RANKL-induced downstream signaling pathways to inhibit osteoclastic differentiation from human osteoclast precursors.

The exact mechanism by which these ADSC-EV affect osteoclast differentiation is not fully clarified. Therefore, to explore the molecular mechanism and biological role of ADSC-EV in modulating osteoclastogenesis, we utilized RNA-seq analysis to find out the RANK downstream signaling pathways modulated by ADSC-EV (FIG. 2A). As shown in FIG. 2B, pathway analysis of these overlapped genes revealed significantly enriched pathways, including osteoclast differentiation and calcium signaling pathway, which ADSC-EV inhibited. Consistent with this, the osteoclastogenic marker genes, such as tumor necrosis factor receptor superfamily member 11A (TNFRSF11A), colony-stimulating factor-1 receptor (CSF1R), triggering receptor expressed on myeloid cells 2 (TREM2), osteoclast-associated receptor (OSCAR), SRC, mitogen activated protein kinase 1 (MAPK1), Calcineurin B-like protein 1 (CBL1), nuclear factor of activated T cell c1 (NFATC1), and Cathepsin K (CTSK), were suppressed by ADSC-EV (FIG. 2C).

In addition, MiR-143 has been reported to inhibit osteoclastogenesis by targeting RANK signaling pathways and is also highly detected in ADSC-EV, suggesting ADSC-EV-containing MiR-143 might involve in the inhibition of osteoclastogenesis (FIG. 2D). To further verify the inhibition of osteoclast activation modulated by ADSC-EV, the osteoclast precursors were treated with ADSC-EV for 3 days. The results revealed that ADSC-EV significantly inhibited RANK, p-SRC, and CBL protein expression signaling downstream. (FIG. 2E). Taken together with FIGS. 1A to 2E, these results imply that ADSC-EV could target RANK and its downstream c-cbl/NFATc1/cathepsin K signaling axis might play pivotal roles in the inhibition of osteoclast formation, which suggests the rationale for ADSC-EV targeting in RANK to inhibit the osteoclastogenesis.

### Example 3

### ADSC-EV Inhibited Receptor Activator of Nuclear Factor Kappa-B Ligand (RANKL) Stimulated Calcium Oscillations

Ca²⁺/calmodulin-dependent kinases (CaMKs)-CAMP response element (CRE)-binding protein (CREB) pathway plays a critical role in RANKL-mediated differentiation and functions of osteoclasts by enhancing the induction of NFATc1 and facilitating NFATc1-dependent gene regulation.

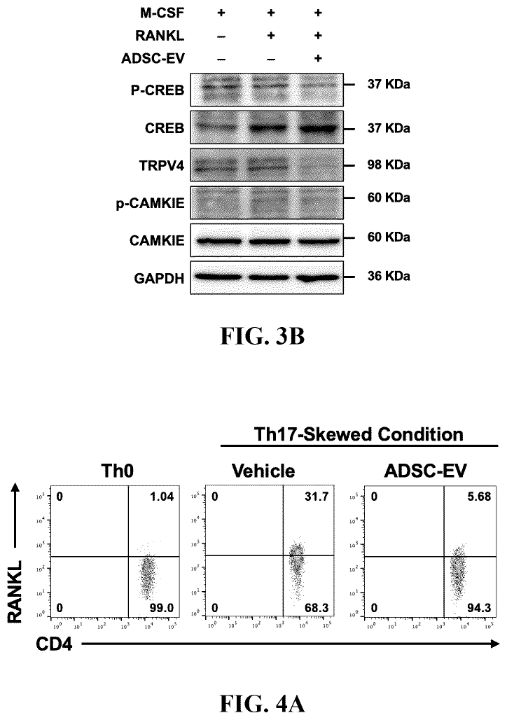

FIGS. 3A and 3B show that ADSC-EV dampened RANKL stimulated calcium oscillations during osteoclast differentiation. (3A) ADSC-EV suppressed Ca²⁺ imaging of RANKL/M-CSF-stimulated osteoclast precursors. The left shows Mag-Fura-4 images of recorded cells analyzed using confocal microscopy (Leica). The right quantifies the Mag-Fura-4 fluorescence ratio in single cells treated with RANKL and ADSC-EV for 120 h. (3B) Protein expression levels of cAMP-response element binding protein (CREB) and p-CREB proteins during osteoclast differentiation in human monocytes treated with RANKL/M-CSF and ADSC-EV for 5 days.

Therefore, to determine whether ADSC-EV mediates osteoclast response to calcium oscillations, we tested the effect of ADSC-EV on calcium oscillations (FIG. 3A). The results showed that the fluorescence intensity of calcium signaling decreased, accompanied by reduced phosphorylation of CREB expression in the osteoclast precursors after treatment with ADSC-EV (FIG. 3B).

### Example 4

### RANKL Expression in the Human T Helper Cell 17 (Th17) Cells and Synovial Fibroblasts was Reduced by ADSC-EV

The osteoclastogenic ability of Th17 cells, a distinctive subset of CD4⁺ T cells, has been attributed mainly to their production of IL-17 and IL-22, which stimulates RANKL expression in synovial fibroblasts in RA joints. In addition, recent studies have highlighted have shown inflammatory joint microenvironment triggers RANKL-inducing properties on RA synovial fibroblasts, which are findings that predict the activation of osteoclasts and structural damage to the affected joints.

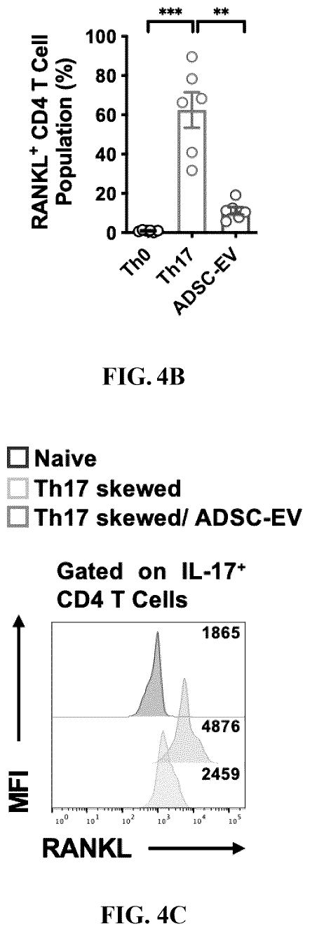

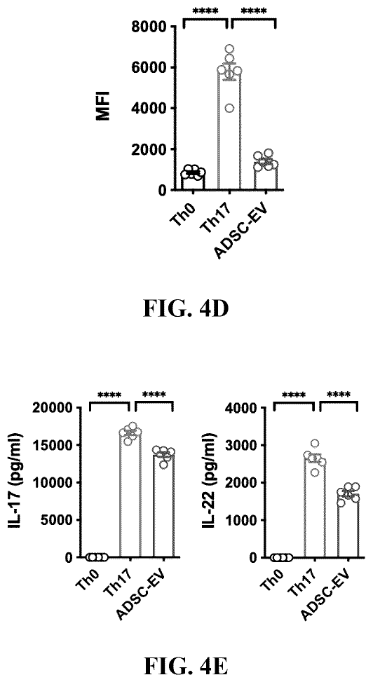

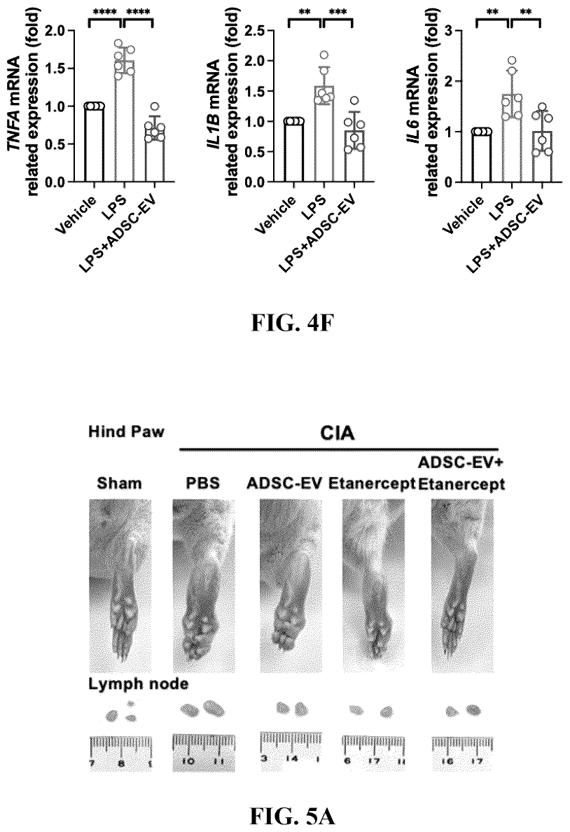

FIGS. 4A-4F show that ADSC-EV significantly suppressed RANKL expression on the human T helper cell 17 (Th17) cells and synovial fibroblasts. (4A) Flow cytometric data showing the percentage of expressing IL-17⁺CD4⁺ populations were significantly decreased by ADSC-EV treatment at day 5. (4B) Quantification of the IL-17⁺CD4⁺ populations by ADSC-EV. (4C) Expression and mean fluorescence intensity (MFI) of RANKL in IL-17⁺CD4⁺ T cells. For MFI and percentages, at least 1000 IL-17⁺CD4⁺ T cells were quantified. (4D) Quantification of the RANKL expression on the IL-17⁺CD4⁺ T populations by ADSC-EV. (4E) IL-17A and IL-22 production were suppressed by ADSC-EV using ELISA assay. (4F) ADSC-EV suppressed genes of inflammation. Analysis of mRNA expression in ADSC-EV treated with LPS-induced synovial fibroblast inflammation. We compared these gene expressions treated with LPS (2 μg/ml) and ADSC-EV (1×10¹¹ particle/ml) for 48 hrs. The cells were harvested, and RNA was extracted to determine gene expression levels of cytokines by using a LightCycler® (Roche Applied Science). The fold change of gene expressions in the y-axis was calculated based on the same gene expression in untreated synovial fibroblasts. One-way ANOVA was performed. * p<0.05, ** p<0.01, *** p<0.001, and **** p<0.0001 for treatments versus Th17 Skewing or indicated groups.

These data revealed that ADSC-EV exhibited a significant reduction in the RANKL expression in the human CD4 cell population under Th17 cell differentiation conditions (FIGS. 4A, 4B). Furthermore, analysis of the RANKL expression showed a significantly decreased mean fluorescent intensity (MFI) of CD4⁺IL-17⁺ cells by ADSC-EV (FIGS. 4C, 4D). Accordingly, ADSC-EV reduced IL-17 and IL-22 protein production of CD4⁺IL-17⁺ cells (FIG. 4E), suggesting ADSC-EV could reduce RANKL responses and inflammatory cytokine production in the skewed Th17 cell differentiation. To confirm the immunomodulatory effect of ADSC-EV-reduced RANKL expression, we examined RANKL induction in synovial fibroblasts in an in vitro LPS-stimulatory inflammation. We found that ADSC-EV significantly suppressed inflammatory cytokine, TNFA, IL1B, and IL6 gene expressions (FIG. 4F).

### Example 5

### A Combination of ADSC-EV and Etanercept Inhibited Inflammation-Driving Bone Resorption in Type II Collagen-Induced Model (CIA) Rats

To evaluate whether reduced bone erosion results from reduced inflammation, we induced acute arthritis using in type II collagen-induced model (CIA) for an inflammatory bone resorption model in rats.

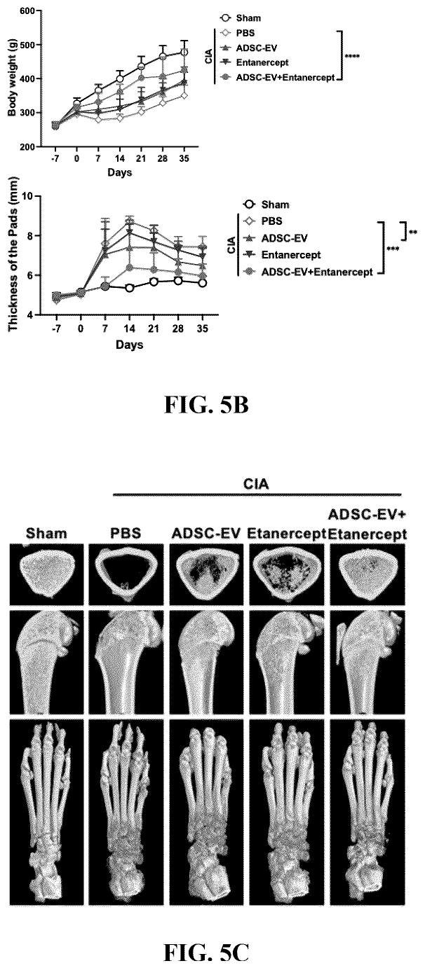

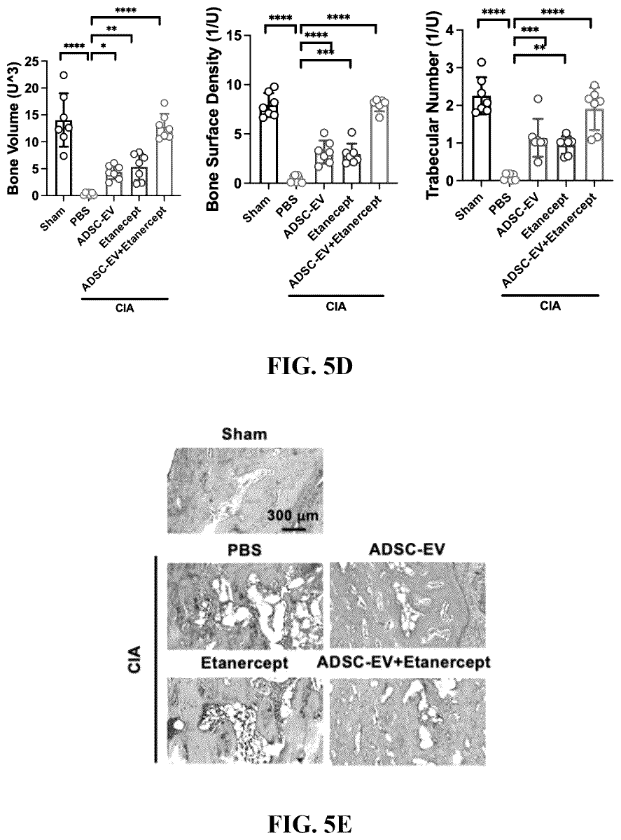

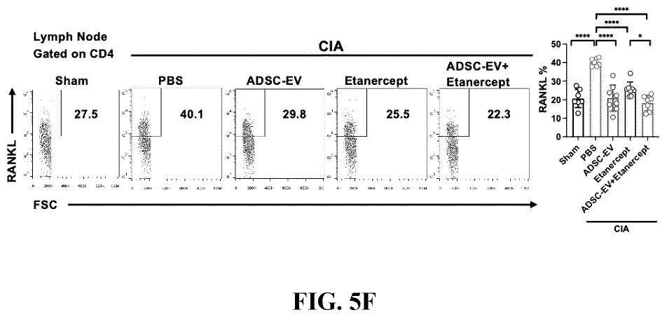

FIGS. 5A-5F show that combination of ADSC-EV and etanercept inhibited bone resorption in type II collagen-induced model (CIA) rats. (5A) Photomicrographs from the hind paw of CIA rats depict severe arthritis and swollen lymph nodes. ADSC-EV inhibited the local swelling of the hindfoot and popliteal lymph nodes in the RA joints. (5B) The body weight and thickness of the pads for each group were by the number of arthritic rats to calculate a mean (±SEM). A combination of ADSC-EV and etanercept reversed the body weight. Hind paw thickness of CIA rats graphs showed a combination of ADSC-EV and etanercept reduced the swelling of the hind paws. (5C) μCT imaging analysis of the hind paws and femurs. Representative three-dimensional μCT reconstruction images showed that ADSC-EV inhibited bone erosion in hind paws and femurs. (5D) The bar charts indicated femoral trabecular bone mass quantification with bone volume, bone surface density, and trabecular number of sham or rats treated with ADSC-EV and suppressed the decrease of bone loss. (5E) TRAP staining of hind paws on paraffin-embedded bone sections in sham, ADSC-EV, and etanercept-treated rats (scale bar, 300 μm; red-purple color to TRAP-positive osteoclasts) are shown on the right. (5F) Lymph nodes of indicated rats were harvested, and cells were analyzed by fluorescence activated cell sorter (FACS). Cells were gated on CD4⁺ cells, and the expression of RANKL was monitored. In addition, the percentage of CD4⁺RANKL⁺ in the live cell gate was determined. Data are given as mean (±SEM) of three independent experiments. n=6.

Of note, we observed hind paw swelling symptoms of arthritis at less severity in ADSC-EV or the clinical use of etanercept administration than in the PBS group of the experimental RA disease (FIG. 5A, upper panel). Furthermore, a combination of ADSC-EV or etanercept treatment showed no apparent clinical signs of arthritis. In line with these data, the swelling of popliteal lymph nodes was reversed by ADSC-EV or etanercept and much more significantly by a combination of ADSC-EV or etanercept treatment (FIG. 5A, lower panel). These observations were also consistent with the reduction of body weight loss after ADSC-EV or etanercept or a combination of ADSC-EV or etanercept treatment (FIG. 5B). Compared to the PBS injection groups in CIA rats as the positive control, ADSC-EV treatment induces not only less bone destruction in hind ankles but also significantly reverses the trabecular bone loss in femurs (FIG. 5C). In striking contrast, a combination of ADSC-EV or etanercept treatment protects against bone loss in bone volume, bone surface density, and trabecular numbers of femoral induced by the arthritic rats (FIG. 5D). These results furthermore demonstrate that ADSC-EV plays a crucial role in preventing inflammatory bone loss. We next explored the therapeutic effect of ADSC-EV in regulating osteoclast activation and would provide greater medical relevance. This CIA rat model allows the investigation of inflammation-induced peri-articular bone resorption during the effector phase of inflammatory arthritis. Administration of ADSC-EV significantly suppressed peri-articular bone erosion and reduced osteoclast numbers and surface in resorption sites (FIG. 5E) compared to the PBS group in CIA rats. In addition, joint swelling was sustained during the 14-day course of inflammatory arthritis, while suppressed bone erosion was observed in the clinical use of etanercept administration, suggesting that coadministration of ADSC-EV with etanercept prominently affects inflammation-driving osteoclastogenesis and bone erosion in this model. This suppression of osteoclast activation and bone resorption are consistent with the CD4 T cells-expressing RANKL expression changes from popliteal lymph nodes in CIA rats (FIG. 5F). These results collectively demonstrate that ADSC-EV suppresses the inflammatory phenotypes, thereby contributing to osteoclastic activation inhibition and bone resorptive suppression.

Biologics are currently known to be used to treat autoimmune diseases, including rheumatoid arthritis. The main side effect is that for hepatitis B carriers or other infectious diseases, the use of the biologics is not recommended, as it may increase viral activity. However, experiments of the present invention confirm that ADSC-EV has significant immune regulation function and can alleviate side effects.

In the experimental design of the present invention, because it is used in response to the clinical use of etanercept, it is only used in living disease animal models. If the evidence for inflammation-induced osteoclast activation is explored, the efficacy of etanercept in the disease model of rheumatoid arthritis can be confirmed. The simultaneous use of ADSC-EV and etanercept has shown its efficacy in inhibiting inflammation.

In summary, the pharmaceutical composition of the present invention achieves the effect on treating arthritis (particularly rheumatoid arthritis) by suppressing osteoclastogenesis, suppressing signaling downstream of RANK, inhibiting RANKL stimulated calcium oscillations, and reducing RANKL expression of human T helper cells and aggressive synovial fibroblasts under inflammation, and the combination of the adipose tissue-derived mesenchymal stem cell (ADSC)-derived extracellular vesicle (EV) and Etanercept inhibits osteoclast differentiation and bone resorption in the subject. It may become a new therapy for inflammation-induced osteoclast activation and inflammatory arthritis in the future.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A pharmaceutical composition for treating arthritis, comprising an adipose tissue-derived extracellular vesicle and a biologic.

2. The pharmaceutical composition according to claim 1, wherein the adipose tissue-derived extracellular vesicle is an adipose tissue-derived mesenchymal stem cell (ADSC)-derived extracellular vesicle (EV).

3. The pharmaceutical composition according to claim 1, wherein the arthritis is rheumatoid arthritis (RA).

4. The pharmaceutical composition according to claim 1, which is in a dosage form for parenteral administration.

5. The pharmaceutical composition according to claim 1, wherein the biologic is Etanercept.

6. A method for treating arthritis, comprising administering to a subject in need thereof a pharmaceutical composition comprising an effective amount of a combination of an adipose tissue-derived extracellular vesicle and a biologic.

7. The method according to claim 6, wherein the adipose tissue-derived extracellular vesicle is an adipose tissue-derived mesenchymal stem cell (ADSC)-derived extracellular vesicle (EV).

8. The method according to claim 6, wherein the arthritis is rheumatoid arthritis (RA).

9. The method according to claim 6, wherein the pharmaceutical composition is in a dosage form for parenteral administration.

10. The method according to claim 7, wherein the adipose tissue-derived mesenchymal stem cell (ADSC)-derived extracellular vesicle (EV) suppresses osteoclastogenesis.

11. The method according to claim 7, wherein the adipose tissue-derived mesenchymal stem cell (ADSC)-derived extracellular vesicle (EV) suppresses signaling downstream of receptor activator of nuclear factor kappa-B (RANK).

12. The method according to claim 7, wherein the adipose tissue-derived mesenchymal stem cell (ADSC)-derived extracellular vesicle (EV) inhibits receptor activator of nuclear factor kappa-B ligand (RANKL) stimulated calcium oscillations.

13. The method according to claim 7, wherein the adipose tissue-derived mesenchymal stem cell (ADSC)-derived extracellular vesicle (EV) reduces RANKL expression of human T helper cells and aggressive synovial fibroblasts under inflammation.

14. The method according to claim 7, wherein the biologic is Etanercept.

15. The method according to claim 14, wherein the combination of the adipose tissue-derived mesenchymal stem cell (ADSC)-derived extracellular vesicle (EV) and the biologic inhibits osteoclast differentiation and bone resorption in the subject.
